(19) United States
(12) Patent Application Publication
(10) Pub. No.: US 2006/0177513 A1
(43) Pub. Date: Aug. 10, 2006

(54) Embolization using poly-4-hydroxybutyrate particles

(75) Inventors: David P. Martin, Arlington, MA (US); Donald Crabtree, Boston, MA (US); Simon F. Williams, Sherborn, MA (US)

Correspondence Address: PATREA L. PABST;PABST PATENT GROUP LLP, 400 COLONY SQUARE, SUITE 1200, ATLANTA, GA, 30361, US

(73) Assignee: Tepha, Inc.

(21) Appl. No.: 11/342,172

(22) Filed: Jan. 27, 2006

Related U.S. Application Data
(60) Provisional application No. 60/648,052, filed on Jan. 28, 2005.

(51) Int. Cl. A61K 31/765; A61K 9/14

(52) U.S. Cl. 424/489; 424/78.37

(57) ABSTRACT

Absorbable particles which comprises poly-4-hydroxybutyrate and/or its copolymers are formulated in injectable suspension suitable for prophylactic or therapeutic embolization, which comprises administering to a human or animal the injectable suspension process for producing particles of the poly-4-hydroxybutyrate and/or its copolymer.

### CROSS-REFERENCE TO RELATED APPLICATION

This applications claims priority to U.S. Ser. No. 60/648,052 entitled “Embolization Using Poly-4-Hydroxybutyrate Particles” filed Jan. 28, 2005 by David Martin, Donald Crabtree, and Simon Williams.

### FIELD OF THE INVENTION

The present invention generally relates to the use of poly-4-hydroxybutyrate and its copolymers in embolization, methods for using these materials in embolization, and processes for producing such materials.

### BACKGROUND OF THE INVENTION

Embolizations (therapeutic vascular occlusions) are used to treat or prevent a range of pathological conditions in situ, including, for example, tumors, vascular malformations, and hemorrhagic processes. They can be performed in a variety of vessels or organs whether healthy or diseased. In these procedures, particulate occlusion agents (emboli) are positioned in the circulatory system using catheters under imagery control. U.S. Pat. No. 6,680,046 to Boschetti reports the following benefits of embolization. In the case of tumors, vascular occlusion can suppress pain, limit blood loss during surgical intervention following embolization or even bring on tumoral necrosis and avoid the necessity for surgical intervention. In the case of vascular malformations, embolization enables the blood flow to the “normal” tissues to be normalized, aids in surgery and limits the risk of hemorrhage. In hemorrhagic events or processes, vascular occlusion produces a reduction of blood flow, which promotes cicatrization of the arterial opening(s). Further, depending on the pathological conditions treated, embolization can be used for temporary as well as permanent objectives.

A range of solid materials, including polyvinylalcohol and polyacrylamide, have been used in embolization procedures. Several patents have also disclosed the combination of some of these materials with imaging and active agents, such as cell adhesion promoters. For example, U.S. Pat. No. 5,635,215 discloses microspheres comprising a hydrophilic acrylic copolymer coated with a cell adhesion promoter and a marking agent, which are useful for embolization. U.S. Pat. No. 5,648,100 discloses an injectable solution for therapeutic embolization, comprising microspheres comprising a hydrophilic acrylic copolymer coated with a cell adhesion promoter and a marking agent, and method of use.

Particles used in embolization should preferably be uniform in shape, and of a defined size range. Notably there have been reports of serious complications when irregular particles have been used in embolization. For example, it has been reported that two infants with symptomatic hepatic arteriovenous malformation died after embolization with polyvinylalcohol particles, and that the heterogeneity of particle size very probably contributed to the death of the infants (see U.S. Pat. No. 6,680,046 to Boschetti).

There is thus a need to develop particles for embolization that are uniform in shape, and have defined size. It is also desirable to develop absorbable particles for embolization that subsequently degrade so that no foreign body is left indefinitely after embolization.

It is therefore an object of this invention to provide a composition for embolization that is degradable in vivo.

It is another object of this invention to provide embolization particles that do not aggregate, can be combined with other components to aid delivery, and/or can incorporate drugs and other agents or actives.

It is yet another object of this invention to provide a method for prophylactic or therapeutic embolization in a human or animal.

### SUMMARY OF THE INVENTION

Methods to produce biocompatible particles of poly-4-hydroxybutyrate or its copolymers for embolization have been developed. These particles are absorbable, unlike currently available embolization particles, and will degrade so that no foreign body is left behind indefinitely after embolization. The particles may comprise other components such as imaging agents, contrast agents, or dyes, cell adhesion factors, anti-angiogenic agents, and/or drugs (that can be eluted and used for example in chemoembolization for the treatment of cancers).

### DETAILED DESCRIPTION OF THE INVENTION

Biocompatible particles for embolization have been developed that are absorbable.

I. Definitions

“Biocompatible” as generally used herein means the biological response to the material or device is appropriate for the device's intended application in vivo. Any metabolites of these materials should also be biocompatible.

“Poly-4-hydroxybutyrate” as generally used herein means a homopolymer comprising 4-hydroxybutyrate units. It may be referred to herein as P4HB, PHA4400 or TephaFLEX™ biomaterial (manufactured by Tepha, Inc., Cambridge, Mass.).

“Copolymers of poly-4-hydroxybutyrate” as generally used herein means any polymer comprising 4-hydroxybutyrate with one or more different hydroxy acid units.

“Absorbable” as generally used herein means the complete degradation of the material over time.

II. Microparticles

Polymers

The particles may be formed from absorbable polymers, such as poly-4-hydroxybutyrate, and copolymers thereof, such as poly-4-hydroxybutyrate-co-3-hydroxybutyrate and poly-4-hydroxybutyrate-co-glycolic acid. Tepha, Inc. of Cambridge, Mass. produces poly-4-hydroxybutyrate and copolymers thereof using transgenic fermentation methods.

Tepha, Inc. (Cambridge, Mass.) produces an absorbable biocompatible biomaterial known as TephFLEX™ (poly-4-hydroxybutyrate), and related copolymers for medical use. Related copolymers include 4-hydroxybutyrate copolymerized with 3-hydroxybutyrate or glycolic acid (U.S. Pat. No. 6,316,262 to Huisman et al., and U.S. Pat. No. 6,323,010 to Skraly et al.), typically in a ratio of up to 30 wt % P4HB. Methods to control the molecular weight of these polymers are disclosed in U.S. Pat. No. 5,811,272 to Snell et al., and methods to purify these polymers for medical use are disclosed in U.S. Pat. No. 6,245,537 to Williams et al. U.S. Pat. No. 6,548,569 to Williams et al. and WO 99/32536 to Martin et al. disclose the degradation rates of these polymers in vivo as well as their use as tissue engineering scaffolds. Other applications of these polymers have been reviewed in Williams, S. F., et al. Applications of PHAs in Medicine and Pharmacy, in Biopolymers, Polyesters, III Vol. 4:91-127 (2002).

Poly-4-hydroxybutyrate belongs to a larger class of materials called polyhydroxyalkanoates, and is usually produced by transgenic fermentation. The polymer cannot be readily synthesized by chemical means with sufficiently high molecular weight for most applications. It is distinguished by its physical and thermal properties, and is degraded in vivo to a natural metabolite (see Martin & Williams, Biochem. Eng. J. 16:97-105 (2003)).

The use of another polyhydroxyalkanoate, poly-3-hydroxybutyrate, formed into spheres of 5-100 μm diameter, for embolization has been reported (see for example, Kassab, A. et al., J. Bioact. Compat. Polym. 14:291-303 (1999)). However, there are no reports of the use of poly-4-hydroxybutyrate in embolization. Notably, although poly-3-hydroxybutyrate and poly-4-hydroxybutyrate belong to the same class of materials, their polymer properties and chemical structures are substantially different. Poly-3-hydroxybutyrate is a rigid brittle material with a melting point around 170° C. derived from a 3-hydroxyacid, whereas poly-4-hydroxybutyrate is derived from a 4-hydroxyacid, and is a strong, flexible and extensible material with a melting point around 60° C. Since it is highly crystalline, the degradation profile of poly-3-hydroxybutyrate is also much longer than that of poly-4-hydroxybutyrate (see Williams, S. F., et al. Applications of PHAs in Medicine and Pharmacy, in Biopolymers, Polyesters, III Vol. 4:91-127 (2002).

In one preferred embodiment, the particles have diameters ranging from 10 μm to 2,000 μm, and are provided in the form of a dry powder or a suspension. The particles may be further sieved into more narrowly defined size ranges, for example, with distributions in sizes between the particles of 0-300 μm, and more preferably 0-200 μm. The size of a prophylactic or therapeutic dose will vary with the nature, type, location and severity of the condition to be treated and the route of administration. It will also vary with age, weight and the response of the patient. An effective amount of particles may range between a few dozen to a few hundred particles, but may be greater or smaller. One skilled in the art may chose to deliver particles of given size ranges, for example, a particle size range of 300-500 μm, 500-700 μm, or 700-900 μm, could be selected for a specific procedure.

The exact size ranges required for each procedure can be readily determined by those skilled in the art.

In another preferred embodiment, the particles completely degrade after two weeks in vivo, more preferably after four weeks in vivo, and even more preferably after 12 weeks in vivo. In one embodiment, the particles comprise between about 0.5% to about 20% poly-4-hydroxybutrate and/or its copolymers by weight.

In yet another preferred embodiment, the particles can be suspended, do not agglomerate prior to use, and can be administered as an injectable suspension with a suitable liquid carrier.

In yet a further preferred embodiment, the particles have a shelf life greater than one year, and more preferably greater than three years. Additionally, a suspension of the particles may have a shelf life exceeding three months, more preferably six months, and even more preferably one year.

Therapeutic, Prophylactic and Diagnostic Agents

In still yet another preferred embodiment, the particles may include a therapeutic, prophylactic or diagnostic or imaging agent. Examples include a dye, imaging agent, contrast agent, cell-adhesion factor, anti-angiogenic agent, and/or drug. Cell adhesion promoters include, but are not limited to, CM dextran, collagen, DEAE dextran, gelatin, glucosaminoglycans, fibronectin, lectins, polycations, and natural biological or synthetic cell adhesion agents. Examples of dyes that can be used to make direct visualization of the particles possible, include, but are not limited to, Cibacron Blue and Procion Red HE-3B. Examples of imaging agents, include, but are not limited to, magnetic resonance imaging agents such as erbium, gadolinium and magnetite. Examples of contrast agents that can be used include, but are not limited to, barium or iodine salts, iodipamide, and amino-3-triiodo-2,4,6-benzoic acid. Non-limiting examples of anti-angiogenic agents that may be incorporated are disclosed in U.S. Pat. No. 6,680,046 to Boschetti. Such components may be incorporated into the particles during their formation, or after their synthesis, for example by grafting or absorption.

II. Methods to Prepare Absorbable Embolization Particles

In a preferred embodiment, the absorbable embolization particles are prepared by an oil in water emulsion technique, as shown in examples 1-7.

In an alternative embodiment, the absorbable embolization particles are prepared by cutting poly-4-hydroxybutyrate filaments into defined lengths, as demonstrated by example 8.

In another alternative embodiment, the absorbable embolization particles may be prepared by extruding the spheres directly by underwater pelletization, or similar process.

The preferred method to sterilize the particles is exposure to ethylene oxide gas. Irradiation (gamma or electron beam) may also be used to sterilize the particles prior to injection into the patient.

III. Methods of Administration of the Absorbable Embolization Particles

The absorbable embolization particles can be suspended, for example, in a physiologically acceptable liquid carrier, such as saline, aqueous solutions, or solutions containing sugars. Notably these liquid carriers may also contain cell adhesion promoters, marking agents, contrast agents, imaging agents, anti-angiogenic agents, or other drugs. The particles may be suspended just prior to use or supplied ready for use. Preferably the suspension is sterile.

Embolization is achieved by administering to a human or animal an injectable suspension comprising an effective amount of the particles, having diameters ranging from about 10 μm to 2,000 μm. The size of a prophylactic or therapeutic dose will vary with the nature, type, location and severity of the condition to be treated and the route of administration. It will also vary with age, weight and the response of the patient. An effective amount of particles may range between a few dozen to a few hundred particles, but may be greater or smaller. One skilled in the art may chose to deliver particles of given size ranges, for example, a particle size range of 300-500 μm, 500-700 μm, or 700-900 μm, could be selected for a specific procedure.

Any suitable route may be used to administer the particles, including for example, parenteral, subcutaneous, or intramuscular, provided that it provides the patient with an effective dose at the desired target or location. The preferred route of administration is to the arteries via a catheter.

Conditions and disease states that can be prevented or treated by embolization include, but are not limited to, solid tumors, vascular malformations, and hemorrhagic events or processes. With respect to tumors, the embolization methods can be used to suppress pain, to limit blood loss occurring during surgical intervention following embolization, or to bring on tumor necrosis and to either avoid or minimize the necessity of surgical intervention. With respect to vascular malformations, the embolization methods can be used to normalize the blood flow to “normal” tissues, to aid in surgery and to limit the risk of hemorrhage. For hemorrhagic events or processes, the embolization methods can be used to reduce blood flow and to promote cicatrization of the arterial opening(s). In addition, the embolization methods can be used as a pre-surgical treatment in order to decrease the blood flow in blood rich organs (e.g., the liver) prior to surgical intervention. Examples of specific conditions that can be prevented or treated by the embolization methods include, but are not limited to, uterine tumors or fibroids; small intestinal hemorrhage, such as that associated with stress ulcer; surgical drain; anastomosis; tuberculous ulcer and nonspecific ulcer; symptomatic hepatic arteriovenous malformation (AVM); primary colorectal cancer; hepatocellular carcinomas; liver metastases; bone metastases; melanomas; cancers of the head or neck; and intracranial meningiomas.

### IV. EXAMPLES

### Example 1

### Poly-4-hydroxybutyrate (P4HB) Microspheres Prepared by an Oil in Water Emulsion Technique from Dilute Polymer Solution

Microspheres of P4HB were made using an oil in water emulsion technique. P4HB (8.4 g, lot # DC04-76-1, M_{w} 494,000 by GPC, Tepha, Inc., Cambridge, Mass.) was dissolved in methylene chloride (304 g, 230 ml) to prepare an 3.7% wt/vol solution. This polymer solution was added slowly with rapid overhead stirring to 2 L beaker containing an aqueous solution (0.5% wt/vol) of polyvinyl alcohol (89% hydrolyzed, M_{w} 31,000-50,000 ). Stirring was done using a 2-inch flat paddle at 820 RPM. The stirring was continued overnight and the methylene chloride was allowed to evaporate from the opened-top beaker. After complete evaporation of the methylene chloride, the stirring was stopped and the microsphere particles were allowed to settle. The supernatant was decanted and the microspheres were resuspended and washed in DI water three times.

The materials and conditions used in the following examples are provided in Table 1.

TABLE 1
Experimental conditions for preparing poly-4-
hydroxybutyrate (P4HB) microspheres.

| | | | CH₂Cl₂ | Stirrer | Vol. | |
|---|---|---|---|---|---|---|
| | | | Final vol. | Speed | 0.5% | Particle |
| Example | 4400 g | CH₂Cl₂ g | ml | (rpm) | PVA | size |
| 1 | 8.4 | 304 | 229* | 820 | 1500 | Small |
| 2 | 38.0 | 300 | 226* | 430 | 1500 | Large |
| 3 | 23.0 | 306 | 231* | 600 | 1500 | Table 2 |
| 4 | 34.5 | 459 | 346* | 595 | 2250 | Table 2 |
| 5 | 23.0 | 306 | 160 | 592 | 1500 | Table 2 |
| 6 | 23.1 | 305 | 185 | 594 | 1500 | Table 2 |
| 7 | 23.1 | 305 | 185 | 700 | 1500 | Table 2 |

*Some evaporation of methylene chloride may have occurred prior to mixing the polymer solution and PVA solution, resulting in a more concentrated solution of P4HB.

### Example 2

### P4HB Microspheres Prepared by an Oil in Water Emulsion Technique from a Concentrated Polymer Solution

Microspheres of P4HB were made using an oil in water emulsion technique as in Example 1 except that a more concentrated solution of P4HB (38 g in 300 g, 226 ml methylene chloride) was used and stirring was done at lower speed (430 RPM) to produce larger P4HB microspheres.

### Example 3

### P4HB Microspheres by an Oil in Water Emulsion Technique from a Concentrated Polymer Solution

Microspheres of P4HB were made using an oil in water emulsion technique as in Example 1 except that a more concentrated solution of P4HB (23 g in 306 g, 231 ml methylene chloride) was used and stirring was done at lower speed (600 RPM) to produce larger P4HB microspheres.

After washing and drying, 14.4 g of microspheres were collected (63% yield). Particles were sized by sieving and sizing data are shown in Table 2.

### Example 4

### P4HB Microspheres Prepared by an Oil in Water Emulsion Technique from a Concentrated Polymer Solution

Microspheres of P4HB were made using an oil in water emulsion technique as in Example 1 except that a more concentrated solution of P4HB (34.5 g in 459 g, 346 ml methylene chloride) was used and stirring was done at lower speed (595 RPM) to produce larger P4HB microspheres. Additionally, a greater volume (2250 ml) of PVA solution (0.5%) was used in a larger 4 L beaker.

After washing and drying of the microspheres, 125.9 g of microspheres were collected (75% yield). Particles were sized by sieving and sizing data are shown in Table 2.

### Example 5

### P4HB Microspheres Prepared by an Oil in Water Emulsion Technique from a Concentrated Polymer Solution

Microspheres of P4HB were made using an oil in water emulsion technique as in Example 1 except that a more concentrated solution of P4HB (23 g in 306 g, 231 ml methylene chloride) was used and stirring was done at lower speed (592 RPM) to produce larger P4HB microspheres.

After washing and drying, 19.0 g of microspheres were collected (83% yield). Particles were sized by sieving and sizing data are shown in Table 2.

### Example 6

### P4HB Microspheres Prepared by an Oil in Water Emulsion Technique from Concentrated Polymer Solution

Microspheres of P4HB were made using an oil in water emulsion technique as in Example 1 except that a more concentrated solution of P4HB (23.1 g in 305 g, 230 ml methylene chloride) was used and stirring was done at lower speed (594 RPM) to produce larger P4HB microspheres.

After washing and drying, 19.94 g of microspheres were collected (86% yield). Particles were sized by sieving and sizing data are shown in Table 2.

### Example 7

### P4HB Microspheres Prepared by an Oil in Water Emulsion Technique from Concentrated Polymer Solution

Microspheres of P4H1B were made using an oil in water emulsion technique as in Example 1 except that a more concentrated solution of P4HB (23.1 g in 305 g, 230 ml methylene chloride) was used and stirring was done at lower speed (700 RPM) to produce larger P4HB microspheres.

After washing and drying, 18.79 g of microspheres were collected (81% yield). Particles were sized by sieving and sizing data are shown in Table 2.

### Example 8

### P4HB Microspheres Prepared from Cut Lengths of Extruded P4HB Fiber

Melt extruded P4HB fiber 275 μm in diameter was cut into lengths of approximately 250 μm to create small particles of P4HB. These particles were less dense than a commercially available contrast agent (RenoCal 76, Bacco Diagnostics) and more dense than 0.9% saline solution but remained suspended in a 50:50 mixture of saline and contrast agent. The particles could be suspended in the solution of contrast and saline and delivered through a 4 F catheter.

TABLE 2
Sizing data for microspheres produced by oil in water emulsion
technique.

| | Weight percent of particles sieved between selected sieves | | | |
|---|---|---|---|---|
| Sample | >500 μm | 500-355 μm | 355-212 μm | <212 μm |
| Example 3 | 1.9 | 11.7 | 58.7 | 27.7 |
| Example 4 | 0.22 | 0.29 | 3.0 | 96.5 |
| Example 5 | 64.1 | 15.2 | 14.3 | 6.5 |
| Example 6 | 65.1 | 19.9 | 11.0 | 4.0 |
| Example 7 | 18.6 | 35.0 | 33.3 | 13.1 |

Modifications and variations of the present invention will be obvious to those skilled in the art from the foregoing detailed description and are intended to come within the scope of the following claims.

We claim:

1. A composition for embolization in a human or animal, comprising particles of poly-4-hydroxybutyrate and/or its copolymers, wherein the particles have a diameter between 10 μm and 2,000 μm, degrade following implantation over a period of time of at least two weeks following implantation.

2. The composition of claim 1 wherein the particles are essentially uniform spheres.

3. The composition of claim 1 wherein the particles are between about 0.5% to about 20% poly-4-hydroxybutyrate by weight.

4. The composition of claim 1 wherein the particles have a size distribution of 300-500 μm, 500-700 μm, or 700-900 μm.

5. The composition of claim 1 wherein the particles have a distribution in sizes between the particles of 0-300 μm, and more preferably 0-200 μm.

6. The composition of claim 1 wherein the particles do not aggregate.

7. The composition of claim 1 further comprising one or more agents selected from the group consisting of therapeutic, diagnostic and prophylactic agents.

8. The composition of claim 7 wherein the agent is a diagnostic agent selected from the group consisting of contrast agents, dyes, and imaging agents.

9. The composition of claim 7 wherein the agent is a therapeutic selected from the group consisting of cell adhesion promoters, anti-angiogenic agents, and drugs.

10. The composition of claim 1 further comprising a pharamaceutically acceptable carrier for intravenous administration.

11. The composition of claim 11, wherein the particles do not clog during administration.

12. A method for prophylactic or therapeutic embolization in a human or animal, comprises administering to the human or animal in need of such embolization, an injectable suspension of the composition of claim 1.

13. The method of claim 12 to prevent or treat a disorder selected from the group consisting of solid tumors, vascular malformations, and hemorrhagic events or processes, including uterine tumors or fibroids; small intestinal hemorrhage, such as that associated with stress ulcer; surgical drain; anastomosis; tuberculous ulcer and nonspecific ulcer; symptomatic hepatic arteriovenous malformation (AVM); primary colorectal cancer; hepatocellular carcinomas; liver metastases; bone metastases; melanomas; cancers of the head or neck; and intracranial meningiomas.

14. The method of claim 12 wherein a tumor is treated to suppress pain, to limit blood loss occurring during surgical intervention following embolization, to bring on tumoral necrosis and to either avoid or minimize the necessity of surgical intervention.

15. The method of claim 12 wherein a vascular malformation is treated to normalize the blood flow to normal tissues, to aid in surgery and to limit the risk of hemorrhage.

16. The method of claim 12 wherein hemorrhagic events or processes are treated to reduce blood flow and to promote cicatrization of arterial openings.

17. The method of claim 12 wherein embolization is used as a pre-surgical treatment in order to decrease the blood flow in blood rich organs prior to surgical intervention.
